(19) United States
(12) Patent Application Publication
(10) Pub. No.: US 2009/0149769 A1
(43) Pub. Date: Jun. 11, 2009

(54) PLAID MOTION RIVALRY FOR DIAGNOSIS OF PSYCHIATRIC DISORDERS

(75) Inventor: John Douglas Pettigrew, Taringa Queensland (AU)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The University of Queensland, St.. Lucia, Queensland (AU)

(21) Appl. No.: 12/295,753

(22) PCT Filed: Apr. 6, 2006
(86) PCT No.: PCT/AU06/00460

§ 371 Date: Feb. 23, 2009

(51) Int. Cl. A61B 5/0476

(52) U.S. Cl. 600/544

(57) ABSTRACT

A method and apparatus for diagnosing a psychiatric disorder or, a predisposition thereto, in a test subject is provided, wherein perceptual rivalry is measured in the test subject during exposure to a moving plaid stimulus. The method may include the step of determining an interhemispheric switch rate of the test subject, and comparing the switch rate with a corresponding reference switch rate to diagnose presence or absence of the psychiatric disorder. Particular psychiatric disorder s include mood disorders and schizophrenia. Also provided is use of the diagnostic method in genetic linkage studies for the identification of the molecular defect(s) underlying a psychiatric disorder, and for the identification of compounds which may alleviate the disorder.

### FIELD OF THE INVENTION

THIS INVENTION relates to diagnosis of psychiatric disorders and/or a predisposition to such disorders. In particular, the invention relates to a method and apparatus for diagnosing schizophrenia and/or mood disorders. The invention also relates to using the diagnostic method for treating psychiatric disorders, for identifying therapeutic compounds for alleviating psychiatric disorders, and for identifying genetic markers associated with psychiatric disorders.

### BACKGROUND OF THE INVENTION

Psychiatric disorders such as schizophrenia and mood disorders (e.g. unipolar depression and bipolar disorder) are relatively prevalent in society and can seriously compromise the ability of sufferers to maintain normal social relationships and a reasonable quality of life.

Early diagnosis and treatment is important. However, this effort is hampered by the fact that psychiatric diagnosis is currently difficult and inaccurate. For example, between 50-70% of “first break” psychoses in young adults that are called “schizophrenia” are subsequently shown to be misdiagnosed.

A particular feature of patients suffering from mood disorders or schizophrenia is that they exhibit rates of perceptual alternation or “switching” that differ from those of non-sufferers. Perceptual alternation occurs where constant visual input gives rise to two or more perceptual interpretations that consecutively alternate between being consciously perceived and suppressed from awareness.

A particular form of perceptual alternation occurs during binocular rivalry, where a different visual stimulus is presented to each eye. This may be distinguished from static, ambiguous images such as the Necker cube and Schroeder's staircase which are viewed by both eyes.

In this regard, diagnosis of schizophrenia and mood disorders through monitoring binocular rivalry are respectively described in International Publication WO 03/026500 and International Publication WO 99/63889.

In the case of mood disorders, a slower rate of interhemispheric switching between an image perceived by one eye and another image perceived by the other eye was associated with the mood disorder, or a predisposition to the disorder.

In the case of schizophrenia, a very high rate of perceptual alternation was observed during binocular rivalry in schizophrenic and schizotypal individuals. A particular manifestation of binocular rivalry, dichoptic stimulus alternation (DSA), occurred at frequencies even higher than a natural frequency of binocular rivalry.

The actual origin and specific nature of the mechanism(s) by which a switch in perception takes place remains in the realm of theory and speculation (Blake & Logothetis 2002, Nat. Rev. Neurosci. 3 13; Leopold & Logothetis 1999, Trends in Cognitive Sciences 3 254; Pettigrew 2001, Brain & Mind 2 85; Tong 2001, Brain & Mind 2 55).

The current consensus appears to be that binocular rivalry represents a series of competitive neural processes and interactions of a multi-level and complex nature (Blake & Logothetis 2002, supra; Crewther et al., In: Binocular Rivalry Eds Alais and Blake. MIT Press 2005; Dayan 1998, Neural Computation 10 1119; Laing & Chow 2002; J. Comput. Neurosci. 12 39; Wilson 2003, PNAS USA 100 14499). These competition-type theories can also been extended to account for the perceptual alternations induced by static ambiguous figures such as the Necker cube and Schroeder's staircase (Gomez et al., 1995, Brain & Cognition 29 103; Laing & Chow 2002, supra; Leopold & Logothetis 1996, Nature 379 549). However it continues to be argued by some authors that binocular rivalry may in fact be mediated by its own separate and distinct neural mechanism (Andrews et al., 2002, Neuroimage 17 890; Meng & Tong 2004, J. Vision 4 539; Tong 2001, supra) at lower levels of the visual cortex (Polonsky et al., 2000, Nature Neurosci. 3 1153).

Another alternative view incorporating the multi-level cortical aspects of rivalry proposes that alternations in perception are triggered by the activity of an underlying interhemispheric oscillatory brainstem network (Pettigrew 2001, supra; Pettigrew & Miller 1998, Proc. Royal. Soc. Lond. B Biol. Sci. 265 2141). Based on a unified extension of previous proposals from multiple sources (Pettigrew & Carter In: Binocular Rivalry Eds Alais and Blake. MIT Press 2005), this theoretical network reportedly includes a number of inter-connected brainstem nuclei with pacemaker activity responsive to modulation by cortical and sub-cortical feedback projections, and is thought to be responsible for the coordination of various and fundamental neurological rhythms such as circadian and ultradian cycles. A variety of supporting evidence exists for this theoretical interpretation of perceptual rivalry (Carter & Pettigrew 2003, Perception 32 295; Funk & Pettigrew 2003, Perception 32 1325; Miller et al., 2000, Current Biology 10 383; Pettigrew & Carter 2002, Adv. Exp. Med. Biol. 508 461), with the most recent demonstrating the involvement of serotonin 1A receptors (implicating a role for the raphe nuclei) in the timing of perceptual alternations (Carter et al., In: Towards a Science of Consciousness 2004. Tucson, Ariz.).

If two sets of gratings with different orientations are superimposed, a plaid pattern is created. When it is moved behind an aperture, the motion of the gratings is ambiguous and when the visual system is confronted with this ambiguity, an apparent oscillation in conscious visual perception is experienced. The unchanging physical motion of the plaid induces stochastic, quasi-regular alternations in conscious perception, between an integrated “diamonds” phase, where a plaid of diamond shaped intersections moves in one direction, and a component “sliding” phase, where the two sets of gratings are seen to slide over each other independently.

Traditionally, plaid stimuli have been studied to investigate the principles of motion integration and segmentation (Grossberg et al., 2001, Vision Res. 41 2521; Hupé & Rubin 2003, Vision Res. 43 531; Mingolla 2003, Neural Networks 16 939; Stoner & Albright 1992, Nature 344 153).

### SUMMARY OF THE INVENTION

The present inventors have surprisingly found that a moving plaid elicits a form of perceptual rivalry distinct from binocular rivalry, referred to herein as “plaid motion rivalry (PMR)”.

Accordingly, the invention broadly relates to use of an ambiguous moving stimulus or image such as a moving plaid, for the diagnosis of a psychiatric disorder, or a predisposition thereto.

The invention also broadly relates to identifying therapeutic compounds for alleviating psychiatric disorders, and for identifying genetic markers associated with psychiatric disorders.

A particularly unexpected aspect of perception during PMR is an asymmetrical hemispheric activation related to the generation of mood states, with the “positive” left hemisphere showing an essentially absolute preference for the diamonds phase.

A measured, relative increase in left hemisphere activity may be associated with an increase in “positive” mood, or a decrease in left hemisphere activity may be associated with a decrease in “positive” mood.

Thus, a particular advantage of the present invention is that the efficacy of anti-psychotic drugs and other treatments can be determined by monitoring their effect upon brain hemisphere bias.

In one broad form, the invention provides a method for diagnosing a psychiatric disorder, or a predisposition thereto, in a test subject including the step of measuring perceptual alternation in the test subject when exposed to an ambiguous moving image to thereby determine whether said test subject has said psychiatric disorder, or has a predisposition thereto.

In one particular aspect the invention provides a method for diagnosing a psychiatric disorder, or a predisposition thereto, in a test subject, said method including the step of: determining an interhemispheric switch rate of the test subject exposed to an ambiguous moving image to thereby determine whether said test subject has said psychiatric disorder, or a predisposition thereto.

Preferably, the method further includes the step of processing each of the signals relating to interhemispheric alternation to convert these signals into digitised signals, and storing the digitised signals for subsequent use.

Preferably, the method further includes the step of comparing the switch rate with a corresponding reference switch rate or switch rate range to diagnose presence or absence of psychiatric disorder or a predisposition thereto.

Suitably, a presence of said psychiatric disorder is diagnosed, or a predisposition thereto is suggested, when the interhemispheric switch rate of the subject is equal to, or within, a corresponding reference switch rate or switch rate range associated with said psychiatric disorder, or a predisposition thereto.

In contrast, an absence of said psychiatric disorder, or a predisposition thereto is discounted, when:
(a) the interhemispheric switch rate of the subject is within a corresponding reference switch rate range associated with one or more normal or control subjects; and/or
(b) the interhemispheric switch rate of the subject is not within a corresponding reference switch rate range associated with said psychiatric disorder, or a predisposition thereto.

In another aspect, the invention provides a method for diagnosing a psychiatric disorder, or a predisposition thereto, in a test subject including the step of measuring perceptual alternation in the test subject when exposed to an ambiguous moving image to thereby determine whether said test subject has said psychiatric disorder, or a predisposition thereto, wherein measurement of perceptual alternation includes measuring respective proportions of duration of activation of left and right brain hemispheres of said test subject exposed to said ambiguous moving image.

In a preferred embodiment, the psychiatric disorder is a mood disorder.

Preferably, a mean duration of left hemisphere activity greater than a mean duration of right hemisphere activity is indicative of a positive mood state.

In another broad form, the invention is directed to a method for treating a psychiatric disorder, or a predisposition thereto, in a test subject exposed to, subjected to or otherwise provided with an ambiguous moving image.

In one particular aspect, the invention provides a method of treating a psychiatric disorder including the steps of:—
(i) determining an interhemispheric switch rate of a patient exposed to an ambiguous moving image;
(ii) comparing said interhemispheric switch rate with a reference interhemispheric switch rate, or switch rate range, associated with said psychiatric disorder to determine a presence or absence of said psychiatric disorder; and
(iii) if said psychiatric disorder is present, treating said patient.

The treatment at step (iii) may include administering to said patient a pharmaceutically-effective dosage of a drug for treating said psychiatric disorder and/or providing psychological counseling, psychotherapy or other non-pharmacological treatments.

Suitably, measuring an interhemispheric switch rate after step (iii) will indicate whether or not said treatment is efficacious.

In another particular aspect, the invention provides a method of treating a psychiatric disorder, or a predisposition thereto, in a test subject exposed to, subjected to or otherwise provided with an ambiguous moving image, said method including the step of: determining and comparing a mean duration of activation of a left brain hemisphere of said patient and a mean duration of activation of a right brain hemisphere of said patient

Preferably, the method includes comparing a mean duration of activation of a left brain hemisphere and a mean duration of activation of a right brain hemisphere of the patient exposed to the ambiguous moving image before and after treating said patient to thereby determine the efficacy of said treatment.

The treatment may include administering to said patient a pharmaceutically-effective dosage of a drug for treating said psychiatric disorder and/or providing psychological counseling, psychotherapy or other non-pharmacological treatments.

Suitably, relatively increased left hemisphere activity measured after treatment indicates that said treatment is efficacious.

In yet another broad form, the invention is directed to an apparatus for diagnosing a psychiatric disorder, or a predisposition thereto, in a test subject exposed to, subjected to or otherwise presented with an ambiguous moving image, said apparatus comprising a display for exposing the test subject to said ambiguous moving image and a means for measuring perceptual alternation in said test subject.

In one particular aspect, the invention provides an apparatus for diagnosing a psychiatric disorder or a predisposition thereto in a test subject, said apparatus comprising:
(a) a display for exposing the test subject to a moving plaid image;
(b) a recorder for monitoring interhemispheric switching in the test subject;
(c) processor for determining an interhemispheric switch rate and for comparing said switch rate with a predetermined data set for providing diagnosis of presence or absence of said psychiatric disorder or a predisposition thereto.

The recorder preferably records a change in perceived image indicated by the test subject.

In a preferred embodiment, the recorder is a computer comprising a digitally-encoded storage medium for storing data and a processor for analysing data.

The change in perceived image may be indicated by the test subject using a subjective device activated by the test subject when a change in stimulus or image is perceived.

Preferably, the subjective device is a keyboard operatively connected to a computer equipped with a digitally-encoded storage medium and a processor for analysing data.

In a further aspect, the invention provides a method for diagnosing a psychiatric disorder, or a predisposition thereto, including the step of using the apparatus described in the aforementioned aspect to determine switching rate in a patient.

In a still further aspect, the invention also provides a method for identifying a candidate therapeutic agent for alleviating, ameliorating or otherwise treating a psychiatric disorder or one or more symptoms thereof, said method including the steps of comparing perceptual alternation in a test subject exposed to an ambiguous moving image or stimulus before and after administration of a candidate therapeutic agent.

In one embodiment, perceptual alternation is compared by measuring interhemispheric switching before and after administration of a candidate therapeutic agent.

In another embodiment, perceptual alternation is compared by measuring relative brain hemisphere activity before and after administration of a candidate therapeutic agent.

In a still yet further aspect, the invention provides a method for identifying one or more genetic markers associated with a psychiatric disorder, or a predisposition thereto, said method including the steps of:
(I) testing respective members of one or more pedigrees affected by said psychiatric disorder, using the method of any one of the above aspects of the invention;
(II) identifying members having said psychiatric disorder; and
(III) conducting genetic linkage analysis on the identified members to identify the or each genetic marker associated with said psychiatric disorder.

In a still yet further aspect, the invention provides a genetic marker identified according to the aforementioned aspect.

According to the aforementioned aspects, the interhemispheric switch rate is determined by measuring a rate of perceptual rivalry in the test subject.

In a preferred embodiment, the ambiguous moving stimulus or image is a moving plaid and the perceptual rivalry is “plaid motion rivalry”.

In particular embodiments, said psychiatric disorder is a mood disorder, inclusive of bipolar disorder and unipolar disorder, or schizophrenia.

Throughout this specification, unless the context requires otherwise, the words “comprise”, comprises” and “comprising” will be understood to imply the inclusion of a stated integer or group of integers but not the exclusion of any other integer or group of integers.

### BRIEF DESCRIPTION OF THE DRAWINGS

In order that the invention may be readily understood and put into practical effect, preferred embodiments of the invention will be described with reference to the accompanying drawings wherein:

FIG. 1 a picture taken of a computer-generated plaid stimulus composed of grey rectangular wave gratings. In the current investigation this stimulus drifted directly upwards behind the circular aperture and the central masking patch and fixation point. Upon viewing this type of dynamic plaid stimulus a perceptual phenomenon arises that we refer to as plaid motion rivalry;

FIG. 2 shows correlated alternation rates for binocular rivalry (abscissa) and for plaid motion rivalry (ordinate). Both perceptual alternations cover a range of around 7×, yet the slowest subjects on binocular rivalry are the slowest subjects on PMR, with similar results for “fast switches” and those in between. N=25, r=0.84, p<0.001. Binocular rivalry rates are consistently higher than the rates for PMR on the high strength apparatus used in this study, but would equalize when using a display with a “weaker” stimulus (e.g. non-moving, or lower contrast);

FIG. 3 shows examples of phase duration frequency histograms from four subjects tested on PMR and binocular rivalry. These demonstrate the inter-individual variation for both stimuli. Above the baseline represents the distribution of intervals (time between perceptual alternations) corresponding to the diamonds phase (PMR) and the horizontal lines phase (binocular rivalry). Below the baseline represents the distribution of intervals corresponding to the opposing phases of PMR (sliding) and binocular rivalry (vertical lines);

FIG. 4 shows a frequency histogram for PMR representing the normalised distribution of the sliding/diamonds phase durations. This distribution is essentially similar to those obtained for binocular rivalry and MIB in normal volunteers. These are expressed as a fraction of the mean phase duration for all subjects. Phase durations approximate a gamma distribution. N=the total number of phase durations, n=number of subjects, R=correlation coefficient. l and r are the values for the parameters that produce the gamma distribution that best approximates the normalised distribution of phase durations;

FIG. 5 shows the relationship between diamonds phase of PMR and self-assessed positive affect as measured by PANAS (N=10, r=0.80, p<0.005). Subjects with the highest positive mood scores experienced the diamonds phase for a relatively greater proportion of the testing time compared with those subjects who scored lower PA;

FIG. 6 shows distinctively similar phase duration frequency histograms for a depressed subject. These demonstrate that longer intervals were recorded for both the sliding phase of PMR and the vertical lines phase of binocular rivalry which may be linked with the right hemisphere and negative affect/depression; and

FIG. 7 shows a schematic depiction of an apparatus used for assessing PMR. The PMR stimulus is displayed on a computer monitor (“display computer”). A subject views the PMR stimulus and presses a predetermined key on the keyboard to indicate a perceptual switch to the diamonds phase and another predetermined key to indicate a change in perception to the sliding phase. The keyboard data is collected by a computer equipped with a data storage medium and a processor for analysing said data (“analysis computer”).

### DETAILED DESCRIPTION OF THE PREFERRED EMBODIMENT

The present invention relates to a test subject viewing two sets of gratings with different orientations that are superimposed to create a plaid pattern which is moved behind an aperture. The inventors surprisingly discovered that this moving plaid pattern elicits a form of inter-hemispheric perceptual rivalry in the test subject, referred to herein as “plaid motion rivalry (PMR)”.

A particularly unexpected aspect of PMR is an asymmetrical hemispheric activation related to the generation of mood states, with the “positive” left hemisphere showing a preference for the diamonds phase of PMR.

Accordingly, increases and decreases in left hemisphere activity can, respectively, be indicative of increases and decreases in positive mood state.

Thus, a particular advantage of the present invention is that if a practitioner uses PMR to see if the correct dose of anti-depressant or other drug or treatment is administered to a patient (for example), since the “positive hemisphere” can be unambiguously identified, an increase in the diamonds phase would unambiguously signal an improvement (i.e. an increase in the relative activity of the left, “positive” hemisphere).

This should be contrasted with a practitioner using prior art rivalry tests to ascertain the correct dose of anti-depressant. In such a case, the practitioner would not know whether the increase in rivalry bias reflected an increase in effectiveness or a decrease in effectiveness of the drug.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

By “psychiatric disorder” is meant any psychiatric disorder or condition which is discernible or diagnosable by measuring perceptual alternation in a person displaying symptoms of said disorder or condition, or in a person predisposed to said disorder or condition.

Such psychiatric disorders include mood disorders, inclusive of bipolar disorder and unipolar depression, and psychotic disorders such as schizophrenia.

By “schizophrenia” is meant a mental disorder with a tendency towards chronicity which impairs functioning and which is characterised by psychotic symptoms involving disturbances of thinking, feeling and behaviour (The American Psychiatric Association's Diagnostic and Statistical Manual, 3rd edition). The condition is suspected to comprise heterogeneous conditions or subtypes.

By “ambiguous optical stimulus” is meant a stimulus or image able to elicit different perceptions which alternate during continued observations of the same stimulus or image. Such ambiguous optical stimuli may be distinguished from binocular stimuli where separate non-ambiguous images are displayed to each eye. The ambiguous optical stimulus may be static (for example, ambiguous figures such as the Necker cube and Schroder staircase) or a moving image referred to herein as an “ambiguous moving image or stimulus”.

A preferred ambiguous moving image or stimulus is a moving plaid.

By “interhemispheric switch rate” is meant the rate of interhemispheric alternation in one or more regions of the brain inclusive of temporo-parietal cortex, hypothalamus, prefrontal, and limbic regions of the brain. Preferably, the interhemispheric switch rate relates to the rate of interhemispheric alternation of the temporo-parietal cortex.

The term “genetic marker” includes within its scope a region of a chromosome, locus, allele or fragment thereof that is associated with a particular phenotype.

### Diagnostic Methods

In particular aspects, the invention provides a method of diagnosing a psychiatric disorder, or a predisposition thereto.

Suitably, the test subject exposed to, subjected to, or otherwise presented with an ambiguous moving stimulus or image.

Preferably, the ambiguous moving stimulus is an image of, or formed by, a moving plaid.

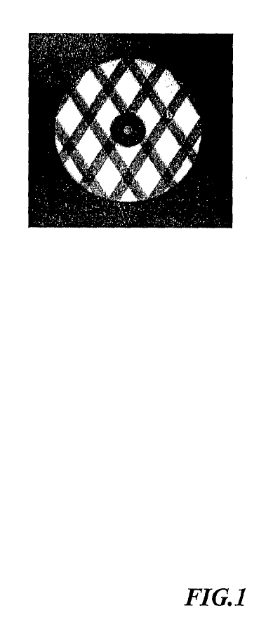

It will be appreciated that FIG. 1 provides but a single example of a moving plaid image. Particular aspects of the plaid image may be varied readily, such as colouring or absence of colour, intensity, speed of movement, thickness of the grating “bars”, grating angle, precise shape and dimensions of the diamonds, background colour and luminence etc. In a similar context, the aperture may be readily varied in relative size/area, shape and speed of movement etc.

The method may include the steps of determining an interhemispheric switch rate of the test subject, and comparing the switch rate with a corresponding reference switch rate to diagnose presence or absence of the psychiatric disorder, or a predisposition thereto.

The interhemispheric switch rate of the subject may be determined by any suitable technique and, in this regard, techniques that indirectly measure a particular interhemispheric switch rate are also contemplated by the invention. For example, an interhemispheric switch rate relating to the temporo-parietal cortex may be determined by measuring the rate of perceptual rivalry (or perceptual alternation) in the subject. Alternatively, an interhemispheric switch rate relating to hypothalamic activity may be determined by measuring rate of alternating sympathetic and parasympathetic activity in the nasal turbinates, also known as the nasal cycle (Shannahoff-Khalsa, 1993, Int. J. Neuroscience 70 285-298).

Suitably, the rate of perceptual rivalry is measured by displaying a moving image (e.g. moving plaid) to the test subject, which image invokes perceptual alternation, signalling respective incidences of perceptual alternation in the test subject during a predetermined period to provide a number of signals and dividing the number of signals by the predetermined period to provide the rate of perceptual rivalry.

Preferably, the method is characterised a subject signals a perceptual alternation or switch. In this context, the subject may signal visually, audibly, or by touch wherein the signal is registrable by a suitable sensor. For example, the subject may depress a button or key that is suitably operably connected to a means for measuring perceptual alternation.

Alternatively, a perceptual alternation may be signalled by a means adapted to measure visually evoked potentials (VEP). In this regard, reference may be made for example, to Brown and Norcia (1997, Vision Res. 37:2401-2408) which teach a real-time, steady-state VEP based on labelling each eye's image with a slightly different temporal frequency so that the record generated by each can be recovered by an electroencephalogram (EEG) by spectrum analysis. In this way, it is possible to track the “waxing” and “waning” of the VEP amplitudes for each eye's image simultaneously during spontaneous rivalry, permitting an analysis of the relative dominance of each eye's image in real-time and to determine alternation rate.

Alternatively, the means may be adapted to monitor eye movement. For example, Blackwood et al (1996, Br. J. Psych. 168 85-92) teach a smooth-pursuit eye tracking procedure in which a subject visually tracks an image and an electro-oculograph is recorded in the horizontal plane via electrodes attached adjacent to the outer canthus of each eye. Reference also may be made to Sweeney et al (1998, Biol. Psychiatry 43 584-594) who disclose the use of infrared recordings to monitor eye movements.

Alternatively, the interhemispheric switch rate may relate to the rate of interhemispheric alternation of hypothalamic activity as mentioned above. Such rate may be determined by measuring the rate of alternating sympathetic and parasympathetic activity in the nasal turbinates, otherwise known as the nasal cycle, as for example disclosed in Shannahoff-Khalsa (1993, supra) and Werntz et al (1983, Human Neurobiol. 2:39-43).

Alternating cerebral hemisphere activation may be determined by EEG recordings as for example disclosed in Shannahoff-Khalsa (1993, supra) and Werntz et al (1983, supra).

Also contemplated, as a measure of interhemispheric switch rate is alternation of performance in hemisphere specific functions such as verbal and spatial abilities (Shannahoff-Khalsa, 1993, supra; Klein & Armitage, 1979, Science 204 1326-1328).

Suitably, the method is further characterised by the step of processing each of the signals relating to interhemispheric alternation to convert these signals into digitised signals, and storing the digitised signals for subsequent use.

Preferably, the step of determining the rate of interhemispheric switching is further characterised by dividing the number of signals corresponding to interhemispheric alternation by the total time the subject is under test. For example, in the case of perceptual rivalry referred to above, the interhemispheric switch rate may be calculated by dividing the number of perceptual switches by the total time of rivalry.

The step of determining interhemispheric switch rate may further include a practice period wherein the subject becomes familiarised with the test. Suitably, this period is not taken into account when determining the rate of interhemispheric switching.

Suitably, a presence of the psychiatric disorder, such as schizophrenia or mood disorder, is suggested, when the interhemispheric switch rate of the subject is within, or equal to, a corresponding reference switch rate or switch rate range associated with the psychiatric disorder.

In such a case, the corresponding reference switch rate may correspond to a predetermined average range of interhemispheric switch rates in subjects having the psychiatric disorder.

In contrast, an absence of the psychiatric disorder may be diagnosed, or a predisposition thereto discounted, when the interhemispheric switch rate of the subject is equal to, or within, a corresponding reference switch rate associated with a normal or control phenotype.

In such a case, the corresponding reference switch rate may correspond to a predetermined average range of interhemispheric switch rates in one or more non-clinical control subjects.

The invention also provides a method for diagnosing a psychiatric disorder, or a predisposition thereto, in a test subject including the step of measuring perceptual alternation in the test subject when exposed to an ambiguous moving image to thereby determine whether said test subject has said psychiatric disorder, or a predisposition thereto, wherein measurement of perceptual alternation includes measuring respective proportions of duration of activation of left and right brain hemispheres of said test subject exposed to said ambiguous moving image.

This is particularly useful for measuring relative left hemisphere activity and positive mood state.

Preferably, the mean duration is a proportion of diamonds phase and sliding phase experienced during PMR, is calculated as a percentage of the total testing time (e.g. proportion of diamonds phase=total diamonds phase time/[total diamonds phase time+total sliding phase time]).

### Mood Disorders

In one particular embodiment, the invention provides a method for diagnosing a mood disorder or a predisposition thereto in a test subject, said method including the steps of:
(i) determining an interhemispheric switch rate of the test subject exposed to a moving plaid stimulus;
(ii) comparing the switch rate with a corresponding reference switch rate, or switch rate range, to thereby diagnose a presence or absence of said mood disorder or a predisposition thereto.

Typically, the interhemispheric switch rate of a test subject having said mood disorder or a predisposition thereto, is slower or less than said reference switch rate.

In such a case, said reference switch rate is associated with a normal or control sample.

In one particular embodiment, said mood disorder is bipolar disorder.

Preferably, a presence of bipolar disorder is diagnosed, or a predisposition thereto is suggested, when the rate of perceptual alternation in the subject is less than 0.40 Hz, preferably less than 0.35 Hz more preferably less than 0.30 Hz or even more preferably less than 0.25 Hz.

It is noted that in relation to bipolar disorder, risk is inversely related to rate. There false positive rate (30%) at 0.45 Hz would fall to zero at 0.20 Hz.

Preferably, an absence of bipolar disorder is diagnosed, or a predisposition thereto is discounted, when the rate of perceptual alternation is greater than 0.40 Hz, preferably greater than 0.45 Hz, more preferably greater than 0.50 Hz and even more preferably greater than 0.55 Hz.

In another particular embodiment, said mood disorder is unipolar disorder.

Preferably, a presence of unipolar disorder is diagnosed, or a predisposition thereto is suggested, when the rate of perceptual alternation in the subject is in the range 0.25 to 0.45 Hz, more preferably 0.30 to 0.45 Hz or even more preferably 0.35 to 0.40 Hz.

Preferably, an absence of unipolar disorder is diagnosed, or a predisposition thereto is discounted, when the rate of perceptual alternation is greater than 0.45 to 0.50 Hz.

It will also be appreciated that the aforementioned ranges include all measurable frequency values therebetween and that a lower or upper limit of one stated range may be a lower or upper limit of any other range or ranges.

The present invention also provides a diagnostic method dependent upon an association between hemispheric activation and mood state, said method including the step of determining respective proportions of duration of activation of left and right brain hemispheres of said test subject exposed to said ambiguous moving image.

According to this aspect, an increased duration of “diamonds phase” or left hemisphere activity may be associated with a positive mood state and therefore be indicative of the absence of depression at the time of testing.

However, it will be appreciated that in the case of bipolar disorder, a bias for the diamonds phase (i.e. left hemisphere activity greater than right hemisphere activity) may also be indicative of a manic state.

Therefore a bias for the diamonds phase, while being associated with a positive mood state in healthy individuals may not necessarily suggest (when considered on its own) the absence of a psychiatric disorder.

Thus, in a preferred embodiment, the invention provides a method of identifying a positive mood state in a test subject, said method including the step of determining mean durations of activation of left and/or right brain hemispheres of a test subject exposed to a moving plaid, wherein a mean duration of activation of a left hemisphere that is greater than a mean duration of activation of a right hemisphere indicates a positive mood state of said test subject.

### Schizophrenia and Schizophrenia Subtype

In another particular embodiment, the invention provides a method for diagnosing schizophrenia, schizophrenia subtype or a predisposition thereto, in a test subject, said method including the steps of:
(i) determining an interhemispheric switch rate of the test subject exposed to a moving plaid stimulus; and
(ii) comparing the switch rate with a corresponding reference switch rate to thereby diagnose a presence or absence of schizophrenia, schizophrenia subtype or a predisposition thereto.

Typically, the interhemispheric switch rate of a test subject having schizophrenia, schizophrenia subtype or a predisposition thereto, is greater than said reference switch rate.

In such a case, said reference switch rate is associated with a normal or control sample.

In embodiments relating to diagnosis of schizophrenia, schizophrenia subtype or a predisposition thereto, an interhemispheric switch rate determined by PMR, indicates a presence of the schizophrenic disorder subtype, or a predisposition thereto, preferably when the rate of perceptual alternation in the subject is greater than 0.60 Hz, more preferably greater than 0.70 Hz, even more preferably greater than 0.80 Hz or more generally in the range 1.0 Hz to 10.0 Hz or 2 Hz to 5 Hz.

Conversely, an absence of schizophrenia or schizophrenic disorder subtype may be diagnosed, or a predisposition thereto discounted, when the rate of perceptual alternation is preferably below 2.0 Hz, more preferably below 1.5 Hz or even more preferably below 1.0 Hz, 0.80 Hz, 0.70 Hz or 0.60 Hz.

### Apparatus for Diagnosis of a Psychiatric Disorder or a Predisposition Thereto

The invention also provides a method of using an apparatus for diagnosing a psychiatric disorder or a predisposition thereto.

Suitably, the apparatus comprises a display for exposing the test subject to a moving plaid stimulus or image and a means for measuring perceptual alternation in said test subject.

In one particular form relating to measurement of interhemispheric switching, the invention provides an apparatus for diagnosing a psychiatric disorder or a predisposition thereto in a test subject, said apparatus comprising:
(a) a display for exposing the test subject to a moving plaid image;
(b) a recorder for monitoring interhemispheric switching in the test subject;
(c) processor for determining an interhemispheric switch rate and for comparing said switch rate with a predetermined data set for providing diagnosis of presence or absence of said psychiatric disorder or a predisposition thereto.

Preferably, the moving plaid stimulus is generated by a display computer having a computer-readable medium that enables the display computer to visually display the moving plaid stimulus or image to the test subject on a monitor operatively linked to said display computer.

The recorder preferably records a change in perceived image indicated by the test subject exposed to the moving plaid stimulus or image.

In a preferred embodiment, the recorder is an analysis computer comprising a digitally-encoded storage medium, or memory, for storage of data and a processor for analyzing said data. The processor suitably includes a timer.

The change in perceived image may be indicated by the test subject using a subjective device activated by the test subject when a change in stimulus or image is perceived.

Preferably, the subjective device is a keyboard operatively connected to a computer equipped with a digitally-encoded storage medium and a processor for analysing data.

The processor is suitably capable of calculating interhemispheric switch rates and/or mean durations of left and right brain hemisphere activation, as hereinbefore described.

In an alternative embodiment, the moving plaid display and data storage/analysis functions could be integrated into a single computer system or network.

### Use of Diagnostic Method to Treat Patients

The invention further provides a method of treating a patient having a psychiatric disorder such as schizophrenia or a mood disorder.

Suitably, said method is capable of alleviating, ameliorating or otherwise treating a psychiatric disorder or one or more symptoms thereof.

Preferably, said method includes the steps of determining an interhemispheric switch rate of the patient when exposed to a moving plaid stimulus, comparing said interhemispheric switch rate with a referenced switch rate or a range of reference interhemispheric switch rates associated with said psychiatric disorder; and administering to said patient a pharmaceutically-effective dosage of a drug for treating said psychiatric disorder when said interhemispheric switch rate indicates that said patient has said psychiatric disorder.

It will be appreciated that PMR testing may be undertaken periodically over the time course of a therapeutic treatment regime, as an additional tool to monitor a patient's progress.

Furthermore, the initial (acute) effects of a therapeutic agent (drug) on PMR switch rate and bias may be different from the (chronic) effects over a time period corresponding to the course of drug therapy.

In the particular case of schizophrenia or schizophrenia subtype, preferably the drug is administered to the patient when the perceptual alternation rate is greater than 1.0 Hz or preferably greater than 2.0 Hz.

In the particular case of bipolar disorder, preferably the drug is administered to the patient when the perceptual alternation rate is less than 0.25 Hz or preferably less than 0.20 Hz.

In the particular case of unipolar disorder, preferably the drug is administered to the patient when the perceptual alternation rate is in the range 0.35 Hz to 0.45 Hz.

As hereinbefore described, a particularly unexpected aspect of PMR is an asymmetrical hemispheric activation related to the generation of mood states, with the “positive” left hemisphere showing a preference for the diamonds phase of PMR.

Accordingly, in one embodiment the invention provides a method for treating a patient in a depressed state including the step of comparing a mean duration of activation of a left brain hemisphere and a mean duration of activation of a right brain hemisphere of a patient exposed to an ambiguous moving image before and after treating said patient to thereby determine the efficacy of said treatment.

It will again be appreciated that PMR testing may be undertaken periodically over the time course of a therapeutic treatment regime, as an additional tool to monitor a patient's progress.

It will also be understood that the initial (acute) effects of a therapeutic agent (drug) on PMR switch rate and bias may be different from the (chronic) effects over a time period corresponding to the course of drug therapy.

Suitably, increased left hemisphere activity, as indicated by a bias towards the “diamonds” phase, indicates that said treatment is efficacious.

In a clinical setting, a practitioner may therefore use PMR to gauge whether a correct dose of anti-depressant or other drug or treatment has been administered to a patient.

This opportunity arises since the “positive hemisphere” can be unambiguously identified. That is, an increase in the diamonds phase would unambiguously signal an improvement (i.e. an increase in the relative activity of the left, “positive” hemisphere) in mood state.

In contrast, a practitioner using prior art rivalry tests to ascertain the correct dose of anti-depressant would not know whether the increase in rivalry bias reflected an increase in effectiveness or a decrease in effectiveness of the drug.

In binocular rivalry, this assignment could be made about 80% of the time, but a fraction of volunteers would show changes in bias that were the reverse and therefore confound any attempt to relate rivalry bias to efficacy of drug (or other treatment).

### Use of Diagnostic Method to Identify Candidate Therapeutic Agents

The invention also provides a method for identifying a candidate therapeutic agent for alleviating, ameliorating or otherwise treating a psychiatric disorder or one or more symptoms thereof, said method including the steps of comparing perceptual alternation in a test subject exposed to an ambiguous moving image or stimulus before and after administration of a candidate therapeutic agent.

In one embodiment, perceptual alternation is compared by measuring interhemispheric switching before and after administration of a candidate therapeutic agent.

In one embodiment relating to mood disorders, the method includes the steps of measuring a first interhemispheric switch rate in a test subject exposed to a moving plaid stimulus, administering or applying a candidate therapeutic agent to said test subject, measuring a second interhemispheric switch rate in the test subject exposed to the moving plaid stimulus, and selecting said candidate therapeutic agent if said second interhemispheric switch rate is greater than said first interhemispheric switch rate.

In one embodiment relating to schizophrenia or schizophrenia subtype, the method includes the steps of measuring a first interhemispheric switch rate in a test subject exposed to a moving plaid stimulus, administering or applying a candidate therapeutic agent to said test subject, measuring a second interhemispheric switch rate in the test subject exposed to the moving plaid stimulus, and selecting said candidate therapeutic agent if said second interhemispheric switch rate is less than said first interhemispheric switch rate.

By “selecting said candidate therapeutic agent” is meant that the candidate therapeutic agent is identified as a potentially useful for treating said psychiatric disorder.

In another embodiment, perceptual alternation is compared by measuring relative brain hemisphere activity before and after administration of a candidate therapeutic agent.

Accordingly, the method includes the step of selecting said candidate therapeutic agent if left brain hemisphere activity (“diamonds phase”) increased after administration of said candidate therapeutic agent.

Suitably, the test subject includes, but is not limited to an animal including a human, brain tissue thereof or brain cell(s) thereof.

Any suitable method may be used to determine interhemispheric switch rate. For example, methods hereinbefore described may be used in the case when the test subject is a human or animal other than human. Alternatively, when the test subject is an animal other than human, electrical activity of brainstem or hypothalamic neurones associated with interhemispheric switching may be measured. This particular technique may also be used when the test subject is brain tissue or brain cells. An example of a method which uses such measurement of electrical activity in vitro and/or in vivo is described by Schaap et al (1997, Brain Res. 753 322-327).

### Use of Diagnostic Method to Identify Genetic Markers Linked to a Psychiatric Disorder or a Predisposition Thereto

Also contemplated is a method for identifying one or more genetic markers associated with a psychiatric disorder.

Preferably, the method includes the steps of testing respective members of one or more pedigrees affected by the psychiatric disorder using the method of the invention, identifying members having the psychiatric disorder or predisposition thereto; and conducting genetic linkage analysis on the identified members to identify the or each genetic marker associated with the psychiatric disorder.

Linkage analysis is well known to those of skill in the art. Exemplary protocols which may be used for this purpose include, but are not limited to, those disclosed in Dracopoli et al (1994, “Current Protocols in Human Genetics”, John Wiley and Sons Inc., USA), Ott, J. (1991, “Analysis of Human Genetic Linkage” Johns Hopkins University Press), and Adams et al. (1998, Am. J. Hum. Genet 62:1084-1091), which are incorporated herein by reference.

The invention also contemplates linkage studies carried out on non-affected individuals i.e. non-pedigree members.

In this regard, one subset of the non-affected individuals will have interhemispheric switch rates differing from those of another subset. The application of linkage analysis to these subsets will be advantageous in identifying molecular markers linked to switch rate (a quantitatively varying trait). These markers may then be employed for the identification of molecular markers linked to the psychiatric disorder.

In an alternative embodiment, one subset of the non-affected individuals will differ according to a mean duration of activation of the left hemisphere. The application of linkage analysis to these subsets will be advantageous in identifying molecular markers linked to switch rate (a quantitatively varying trait). These markers may then be employed for the identification of molecular markers linked to the psychiatric disorder.

In a particular embodiment, the mean duration is a proportion of diamonds phase and sliding phase experienced during PMR, calculated as a percentage of the total testing time (e.g. proportion of diamonds phase=total diamonds phase time/[total diamonds phase time+total sliding phase time]).

The invention also extends to the genetic marker(s) obtained by the aforementioned process.

In order that the invention may be readily understood and put into practical effect, particular preferred embodiments will now be described by way of the following non-limiting examples.

### EXAMPLES

The aim of the first part of this investigation was to address the question as to whether the timing of alternations induced by binocular rivalry are correlated with those experienced during plaid motion rivalry. It will also be established if a similar wide range of inter-individual variation in perceptual alternation rates exist for PMR as there does for binocular rivalry. The results have demonstrated that the underlying switch that triggers the alternation in all forms of rivalry (BR and PMR) results from a common mechanism.

Furthermore, this investigation was designed to the inventor's proposal of a link between phase of rivalry and lateralized affective and cognitive processes

The second part of this investigation focused on the possibility that subjects' mood state may be reflected in the pattern of perceptual alternations they experience during PMR.

### Example 1

### The Temporal Dynamics of PMR and Binocular Rivalry

Experiments were conducted to determine each subject's alternation rate for PMR (within a constant set of plaid parameters) and for binocular rivalry. For example, for a subject tested on binocular rivalry the duration of time between each perceptual switch was measured. The total number of perceptual switches was then divided by the total viewing time in order to calculate an alternation rate (Hz).

Additionally, when normalised across subjects the stochastic nature of all perceptual rivalries studied to date generally reveals phase interval durations that approximate a gammalike distribution (Borsellino et al., 1972, Kybernetik 10 139; Carter & Pettigrew, 2003, supra; Fox 1967, Perception & Psychophysics 2 432; Kleinschmidt et al., 1998, Proc. Royal. Soc. Lond. B Biol. Sci. 265 2427; Muckli et al., 2002 J. Neurosci 22 RC219). As a matter of convention we also therefore determined the distribution of normalised PMR phase durations for the sample of subjects which, along with alternation rates and subject frequency histograms were automatically calculated using a commercial software programme with data collection and analysis capabilities (BiReme Systems: www.bireme.com.au).

### Materials and Methods

### Subjects

The study recruited a total of 29 subjects aged between 16 and 55 years, and included members of the public as well as students and staff from the University of Queensland. Four subjects' data were excluded from further analysis; 3 failed to show the required visual acuity (6/9 or better) and a fourth continually perceived PMR as a rotating sphere, rather than alternating between the two common perceptual interpretations. The remaining 25 subjects were naïve as to the aims and hypotheses of the experiment although five were experienced at psychophysical experiments and demonstrated a degree of knowledge and awareness of perceptual rivalry. The University of Queensland Behavioural and Social Sciences Ethical Review Committee approved the study.

### Apparatus and Stimuli

A plaid stimulus demonstration at the following website: http://www.cns.nyu.edu/˜hupe/arvo01demo/index.html)

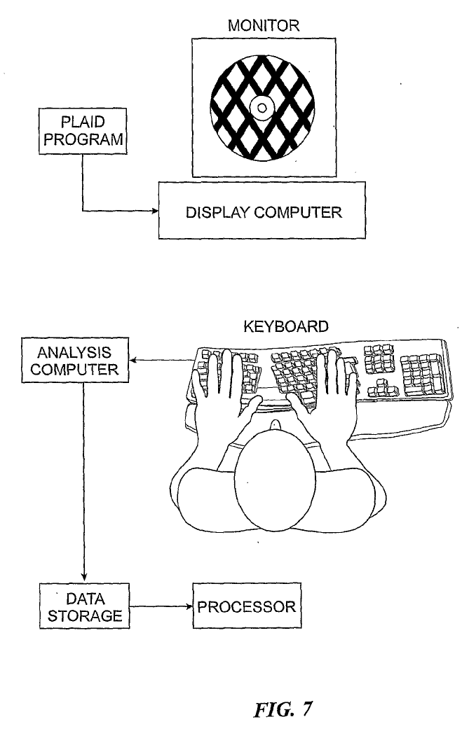

A schematic depiction of a preferred embodiment of a PMR apparatus is shown in FIG. 7. The PMR stimulus (also seen in FIG. 1) was generated using the Plaid_Program 2002, kindly provided by Jean-Michel Hupe and Nava Rubin. The stimulus was displayed on a Personal Computer (PC Gateway EV500) with an 8×11 inch colour monitor (“display computer”).

Subjects viewed the stimulus from a distance of 57 cm and data was collected using a commercial collection programme (BiReme Systems; www.bireme.com.au) run on an adjacent PC (“analysis computer”).

Alternatively, the moving plaid display and data storage/analysis functions could be integrated into a single computer system or network.

The PMR stimulus consisted of symmetric rectangular-wave gratings forming a plaid that moved in a global direction of 90° (upwards). The speed of the gratings was 3°/second. The angle between the gratings' directions of motion was 115° and the entire stimulus moved behind a centrally located circular aperture with a diameter of 8°. The area outside the aperture was dark grey and filled the remaining screen.

The gratings consisted of grey bars superimposed on a white background. The duty cycle of the gratings, calculated as the width of the grey bars divided by the total cycle (total cycle=width of the grey bars plus the width of the inter-bar region) was 30%. The diamond intersection regions of the gratings were in the transparency range (i.e. darker grey relative to the bars—see FIG. 1) which induces the illusion of transparency (Stoner et al., 1990, Nature 344 153) when the gratings are seen to slide over each other during the sliding phase of perception. A central red fixation point (diameter=0.4°) was overlaid on a dark grey masking patch (diameter=2°) designed to reduce optokinetic nystagmus eye-movements (Hupé & Rubin 2003, supra).

The binocular rivalry stimulus (consisting of green vertical and horizontal lines) was generated using a VisionWorks package and displayed on a green monochrome, 12×16 inch computer monitor (P46 phosphor, persistence=500 nsec). To simultaneously project a different image to each eye, the vertical and horizontal lines alternated in rapid succession (120 Hz) and were viewed from a distance of 3 m through NuVision liquid crystal shutters. The shutters alternated in synchrony with the stimulus such that the left eye only viewed vertical lines and the right eye horizontal lines. Data was collected using the same collection programme (BiReme Systems”) run on an adjacent PC. The stimulus consisted of drifting green vertical and horizontal square-wave gratings overlaid on a black background. The vertical gratings moved from left to right and the horizontal gratings drifted upwards.

The entire stimulus moved behind a circular aperture and subtended 1.5° of visual angle with a spatial frequency of 8 cycles/degree moving at 4 cycles/second.

### Testing Procedure

Each subject was tested on PMR and on binocular rivalry with a 5-minute break between each rivalry test. It was not possible to study both rivalries simultaneously, both for technical reasons and because of the problem of attending simultaneously to two processes. All experiments were conducted in a dimly lit room and the order of testing was counterbalanced. For both PMR and binocular rivalry, subjects were given an initial familiarisation and instruction period of approximately 1-2 minutes after which data was collected over four 100-second trials, with a 30-second break between each trial. A 5-second warning was given to subjects by the collection programme prior to the beginning of each trial.

During binocular rivalry testing, subjects indicated their perceptual switches by pressing one of two keys on a standard keyboard (V or B). The V key was highlighted by a small perspex disk with a vertically orientated ridge, while the B key had a small perspex disk with a horizontally orientated ridge. Subjects were instructed to press the V key when their perception changed to mostly vertical lines and when their perception switched to horizontal lines they were instructed to press the B key. As described in previous studies using this collection programme (Carter and Pettigrew 2003, supra; Pettigrew & Miller 1998, supra), subjects were given the option of pressing the space bar to indicate periods of mixed/intermediate perception, which the programme automatically removed prior to analysis. This option was used minimally by nine of the participating subjects.

For PMR, subjects were instructed to press the B key to indicate a perceptual switch to the diamonds phase and when their perception changed to the sliding phase they were instructed to press the V key. When subjects were given a 5-second warning by the collection programme they were instructed to view the plaid and maintain fixation. This was found to successfully counteract the temporal phenomenon unique to the perception of plaids consisting of rectangular wave gratings (Hupé & Rubin, 2003, supra) found that for a temporally isolated trial the initial percept generally always consists of the diamonds phase and the time of this interval is longer than subsequent diamond phase intervals). None of the subjects used the space bar option (to indicate periods of mixed/intermediate perception) during PMR.

### Results and Discussion

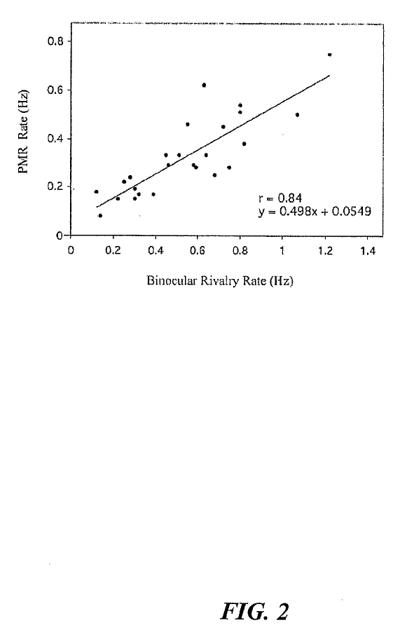

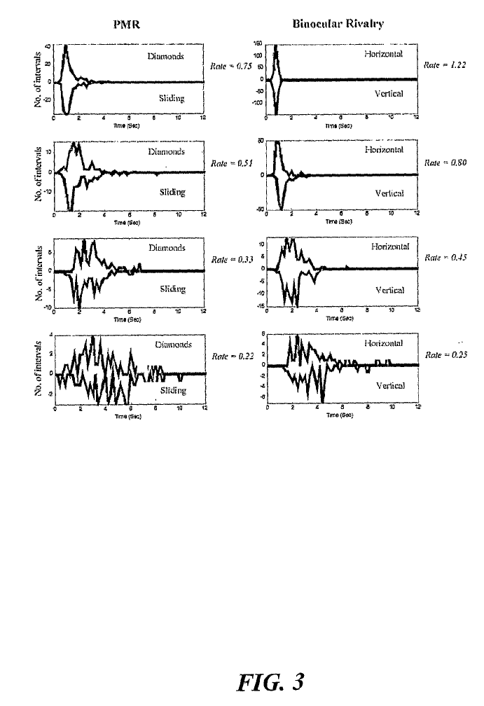

The data described herein have led to the present inventors' proposal that all perceptual rivalries are mediated by a common switching mechanism. The inventors found a significant inter-individual correlation between alternation rates induced by binocular rivalry and PMR (Pearson's correlation coefficient, r=0.84, p<0.001) (FIG. 2). Additionally there was a diverse range of rates recorded for both PMR (0.08 Hz-0.75 Hz) and binocular rivalry (0.14 Hz-1.22 Hz). Subject frequency histograms illustrating the distribution and durations of perceptual phase intervals (FIG. 3) clearly demonstrate the inter-individual variation across the two stimuli.

The majority of subjects (96%) recorded slower rates on PMR.

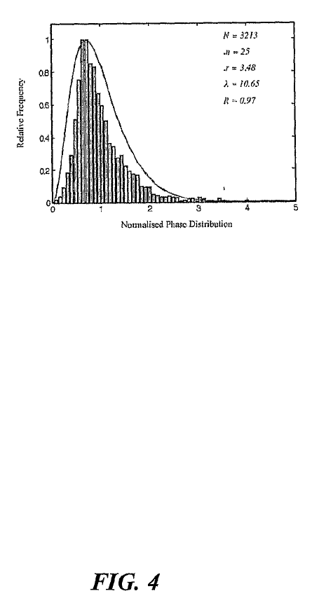

The mean rates for PMR and binocular rivalry were 0.33 Hz (±0.03) and 0.54 Hz (±0.06) respectively. An analysis of variance found these to be significantly different (F 1, 24=11.60, p<0.002). Normalising the PMR data for each subject generated a frequency histogram that approximates a gamma-like distribution (r=0.97) (FIG. 4). This distribution is essentially similar to those obtained for binocular rivalry (r=0.94) and MIB (r=0.96) in prior experiments (Carter & Pettigrew 2003, supra).

### Example 2

### The Relationship Between Mood and PMR Phases

The brainstem oscillator theory for rivalry is also sometimes referred to as the interhemispheric switching hypothesis (Miller et al., 2000, supra; Pettigrew & Miller 1998, supra).

In broad terms this original formulation suggests that part of the oscillator network (at the level of the ventral tegmental area) may function to facilitate a switch between each hemisphere's complementary viewpoint or cognitive style, and that relative hemispheric activity can bias the oscillator via feedback projections (Pettigrew 2001, supra).

This aspect of the theory, which can be interpreted in a variety of ways was partly derived from Ramachandran's formulation (Ramachandran, 1994, Int. Rev. Neurobiol. 37 291; Ramachandran, 1995, Consciousness & Cognition 4 22) outlining the complexity of hemispheric interactions and their respective cognitive styles. This formulation emphasizes the complementary roles of the two hemispheres: the integrative, hypothesis-driven style of the left hemisphere tends to deny discrepancies that interfere with its globally consistent, goal-orientated interpretation; whereas the veridical, discrepancy-detecting style of the right hemisphere, often referred to as an anomaly detector (Ramachandran, 1995, supra; Smith et al., 2004, Brain & Cognition 55 458; Smith et al., 2002, Brain & Cognition 48 574) is involved in actively monitoring all available information in order to highlight those discrepancies and bring them into conscious awareness.

The brainstem perceptual oscillator is therefore thought to facilitate a switch between functionally homologous regions of each hemisphere that are responsible for the formulation of alternative, but complementary perceptual interpretations, encompassing both cognitive and affective aspects of perception. With respect to plaid perception and based upon Ramachandran's formulation, the diamonds phase consisting of a global integrated pattern of diamonds moving in a single direction would more likely be associated with the left hemisphere whereas the bi-directional and separated percepts of the complementary phase might be considered to be more in line with the discrepancy detecting style of the right hemisphere.

For binocular rivalry, hemispheric stimulation and disruption experiments (Miller et al., 2000, supra) have linked the horizontal lines phase with the left hemisphere and vertical lines with the right, however for a few volunteers these associations were found to be reversed. In general, due to a cultural bias for horizontally orientated human scripts the left hemisphere's association with horizontal lines may be related to its predisposition for language processes and sentence reading (Pettigrew 2001, supra)

One way to test such predictions for PMR is to investigate the relationship between rivalry phases and accepted hemispheric assignment (Pettigrew & Carter 2002, supra), such as the lateralization of positive and negative mood states (Davidson 2001, Ann. NY Acad. Sci. 935 191; Davidson et al., 1990, J. Personality & Soc. Psychology 58 330).

For example, studies have shown that self-assessed positive and negative affect measures reliably correlate with anterior hemispheric activation asymmetry, linking a positive mood state with a dominance of activation in the anterior regions of the left hemisphere, and a negative mood state corresponding to the homologous areas of the right hemisphere (Tomarken et al., 1992, J. Personality & Soc. Psychology 62 676). Using mood measures to investigate such linkages for plaid motion rivalry will firstly determine if perception of this stimulus reflects mood state and secondly reveal the possibility that hemispheric assignment exists for the opposing perceptual phases of PMR.

### Materials and Methods

### Subjects and Procedure

Ten subjects participating in Example 1 agreed to take part in this additional procedure involving the self-assessment of positive and negative mood variables.

Following binocular rivalry and PMR testing, subjects completed the Positive and Negative Affect Schedule (PANAS; Watson et al., 1988, J. Personality & Soc. Psychology 54 1063). The PANAS is a simple form consisting of 10 positive adjectives (for example interested, excited, inspired) and 10 negative adjectives (for example upset, hostile, scared) that subjects rated on a scale of 1-5 (1 indicating the least intense and 5 corresponding to the most intense) according to how they felt during the then present moment. Each subject's PANAS scores were calculated by adding their ratings to give an overall positive affect (PA) and a negative affect (NA) score of between 10 and 50. The proportion of diamonds phase and sliding phase experienced during PMR was calculated as a percentage of the total testing time (e.g. Proportion of diamonds phase=total diamonds phase time/[total diamonds phase time+total sliding phase time]).

The same calculation was also performed for the binocular rivalry data.

### Results and Discussion

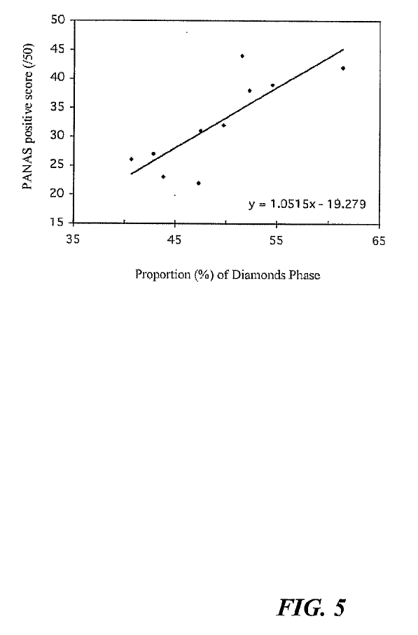

A significant positive correlation was found to exist between positive affect scores and the proportion of time subjects experienced the diamonds phase of PMR (Pearson's correlation coefficient, r=0.80, p<0.005) (FIG. 5). Subjects therefore with the highest degree of self-assessed positive affect experienced the diamonds phase of PMR for a significantly greater proportion of the testing time compared with those subjects who reported lower PA scores. No relationship was found between negative affect scores and the proportion of time subjects experienced the sliding phase of PMR, however this was not surprising given the consistently low (mean score out of 50 was 14.4) and poor range of NA scores (11-20) recorded.

This is the first time that coherent, global or integrated motion (diamonds phase) as a form of rivalry has been shown to share a relationship with positive mood state. Accordingly, perception during PMR would seem to be linked with asymmetrical hemispheric activation related to the generation of mood states, with the “positive” left hemisphere showing a preference for the diamonds phase. This linkage between the diamonds phase and the left hemisphere is also consistent with predictions based upon Ramachandran's formulation of the complementary cognitive styles of each hemisphere. The generation of alternative, complementary perceptual phases during PMR therefore follows theoretical predictions consistent with the interhemispheric switching hypothesis.

No relationship was found between affect scores and binocular rivalry phases when subjects were pooled as a group. Recent findings suggest binocular rivalry is only weakly influenced by top-down attentional processes compared to other forms of rivalry (Meng & Tong 2004, supra; van Ee et al., 2005, Vision Res. 45 41), but there may be little connection between top-down attention effects and the mood-related bias for one phase, because MIB shows a strong mood-related correlation but is virtually immune to top-down attentional changes (Carter & Pettigrew 2003, supra).

A likely factor in the failure to show a clear mood-related effect in the group is that individual volunteers can show perceptual changes in opposite directions, an effect explained in terms of differing individual hemispheric assignment for simple stimuli like contours (Miller et al., 2000, supra). Strongly asymmetrical rivalries like PMR and MIB may not be subject to the same degree of inter-individual variation. Within subject design, where the volunteer is used as his/her own control, show clear mood-related effects on binocular rivalry.

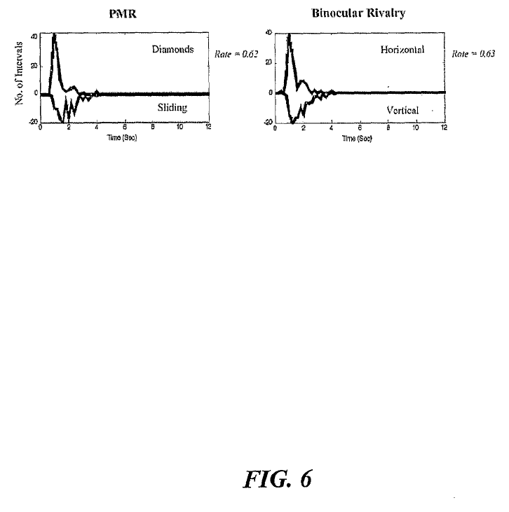

To demonstrate that binocular rivalry can indeed show mood-related effects similar to those seen in other forms of rivalry, we would like to highlight the results of one of our volunteers whose pattern of perceptual alternations on both binocular rivalry and PMR were tightly coupled and appear to have been influenced by lateralized mood-related processes in a remarkably similar way. The subject's frequency histograms are displayed in FIG. 6. It can be seen how longer intervals were recorded for the vertical lines phase of binocular rivalry and the sliding phase of PMR, resulting in very similar perceptual biases for these two phases.

It has previously been reported that depressed subjects display a marked bias for the vertical lines phase (reportedly linked with the right hemisphere) when tested on binocular rivalry (Pettigrew 2001, supra). Interestingly, upon subsequent interactions with the subject, this young and healthy individual with no history of mental illness revealed that they were suffering from a mild episode of depression. Unfortunately when they were able to return to the lab two months later their depressive episode had persisted (to the extent that they were subsequently diagnosed and had commenced antidepressant medication), and upon retesting they again recorded similarly polarised perceptual biases, with a predominance of the vertical lines phase of binocular rivalry and the sliding phase of PMR (results not shown).

Depression is a complex condition (Rotenberg 2004, Progress in Neuro-Pharmacol. & Biol. Psych. 28 1) and not simply a transient negative mood, however it has been associated with greater relative anterior activation of the right hemisphere (Davidson et al., 2002, supra; Tucker et al., 1981, Arch. Gen. Psych. 38 169). In light of this association, the subject's perceptual bias for the sliding phase and vertical lines phase is in accordance with both the cognitive style of the right hemisphere and the theoretical predictions of the interhemispheric switching hypothesis. Moreover, this result provides a clear demonstration of how patterns of perceptual alternations during binocular rivalry can also be similarly reflective of mood state.

### CONCLUSION

For the group of subjects tested during this investigation there was a wide range of inter-individual variation in the timing of perceptual alternations induced by both binocular rivalry and plaid motion rivalry. Due to differences in stimulus strengths and characteristics, rivalry rates were slower for PMR compared with those recorded for binocular rivalry, however rates were significantly correlated across both phenomena. This suggests that while different visual/perceptual processes and neural substrates may contribute to the various perceptual phases of binocular rivalry and PMR, these processes and substrates appear to be subject to a common switching mechanism. These results are compatible with a number of theories for rivalry.

The second major finding of this investigation was that the diamonds phase of PMR, consisting of global, coherent or integrated motion appears to share a relationship with positive mood state. For one subject both a predominance of the sliding phase of PMR and the vertical lines phase of binocular rivalry appeared to demonstrate a clear association with a depressed mood state. These results provide further evidence for an emerging new dimension to the phase aspects of rivalry and present a challenge to competition theorists whose models focus primarily on processes within the visual hierarchy. While competition theories may account for the correlated frequency aspects of rivalry, such theories are yet to address how lateralized mood-related processes might distinctively influence the temporal pattern of rivalry phases. Overall the results of this investigation are consistent with theoretical predictions of the brainstem oscillator theory and its interhemispheric switching hypothesis.

While the data presented herein primarily addresses the use of PMR for diagnosis and treatment of mood disorders it will nevertheless be appreciated that it may readily be extendable to a broad spectrum of psychiatric disorders including but not limited to schizophrenia and schizophrenia subtypes, for example.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

The disclosure of each patent and scientific document, computer program and algorithm referred to in this specification is incorporated by reference in its entirety.

1. A method for diagnosing a psychiatric disorder, or a predisposition thereto, in a test subject including the step of measuring perceptual alternation in the test subject when exposed to an ambiguous moving image to thereby determine whether said test subject has said psychiatric disorder, or a predisposition thereto.

2. The method of claim 1, wherein perceptual alternation is measured by determining an interhemispheric switch rate of the test subject exposed to the ambiguous moving image.

3. The method of claim 2, further includes the step of comparing the switch rate with a corresponding reference switch rate to diagnose presence or absence of psychiatric disorder or a predisposition thereto.

4. The method of claim 3, wherein said psychiatric disorder is diagnosed, or a predisposition thereto is suggested, when the interhemispheric switch rate of the subject is within a corresponding reference switch rate associated with said psychiatric disorder, or a predisposition thereto.

5. The method of claim 3, wherein an absence of said psychiatric disorder is determined, or a predisposition thereto discounted, when the interhemispheric switch rate of the subject is within a corresponding reference switch rate associated with said psychiatric disorder, or a predisposition thereto.

6. The method of claim 4, wherein the psychiatric disorder is a mood disorder.

7. The method of claim 6, wherein the mood disorder is bipolar disorder.

8. The method of claim 7, wherein the corresponding reference switch rate range is less than 0.40 Hz.

9. The method of claim 5, wherein the psychiatric disorder is schizophrenia.

10. The method of claim 9, wherein the corresponding reference switch rate range is greater than 1.0 Hz.

11. The method of claim 1, said method including the step of determining respective proportions of duration of activation of left and right brain hemispheres of said test subject exposed to said ambiguous moving image.

12. The method of claim 11, wherein the psychiatric disorder is a mood disorder.

13. The method of claim 12, wherein a mean duration of left hemisphere activity greater than a mean duration of right hemisphere activity is indicative of a positive mood state.

14. The method of claim 1, wherein the ambiguous moving image is a moving plaid.

15. A method for treating a psychiatric disorder in a patient, said method including the steps of:
(i) measuring perceptual alternation in the test subject when exposed to an ambiguous moving image, to thereby determine whether said test subject has said psychiatric disorder, or a predisposition thereto; and
(ii) if said psychiatric disorder is present, treating said patient.

16. The method of claim 15, wherein step (i) includes the steps of:
(a) determining an interhemispheric switch rate of said patient;
(b) comparing said interhemispheric switch rate with a corresponding reference interhemispheric switch rate or switch rate range associated with said psychiatric disorder to determine a presence or absence of said psychiatric disorder.

17. The method of claim 16, wherein the psychiatric disorder is a mood disorder or schizophrenia.

18. The method of claim 15, wherein the ambiguous moving image is a moving plaid.

19. A method for treating a patient in a depressed state including the step of comparing a mean duration of activation of a left brain hemisphere and a mean duration of activation of a right brain hemisphere of a patient exposed to an ambiguous moving image before and after treating said patient to thereby determine the efficacy of said treatment.

20. The method of claim 19, wherein the ambiguous moving image is a moving plaid.

21. The method of claim 20, wherein a relative increase in left brain hemisphere activity indicates said treatment is efficacious.

22. An apparatus for diagnosing a psychiatric disorder or a predisposition thereto in a test subject, said apparatus comprising:
(a) a display for exposing the test subject to an ambiguous moving image; and
(b) means for measuring perceptual alternation in said test subject.

23. The apparatus of claim 22, wherein said means for measuring perceptual alternation comprises:
(a) a recorder for monitoring interhemispheric switching in the test subject; and
(b) a processor for determining an interhemispheric switch rate and for comparing said switch rate with a predetermined data set for providing diagnosis of presence or absence of said psychiatric disorder or a predisposition thereto.

24. The apparatus of claim 23, wherein the recorder records a change in perceived image indicated by the test subject.

25. The apparatus of claim 23, wherein the recorder is a computer comprising a digitally-encoded storage medium and a processor.

26. The apparatus of claim 22, wherein a change in perceived image is indicated by a subjective device activated by the test subject.

27. The apparatus of claim 26, wherein the subjective device is a keyboard operatively linked to said recorder.

28. A method for identifying a candidate therapeutic agent for alleviating, ameliorating or otherwise treating a psychiatric disorder or one or more symptoms thereof, said method including the steps of comparing perceptual alternation in a test subject exposed to an ambiguous moving image or stimulus before and after administration of a candidate therapeutic agent.

29. The method of claim 28, wherein perceptual alternation is compared by measuring interhemispheric switching before and after administration of said candidate therapeutic agent.

30. The method of claim 29, wherein perceptual alternation is compared by measuring relative brain hemisphere activation before and after administration of said candidate therapeutic agent.

31. The method of claim 28, wherein the test subject is a mammal.

32. The method of claim 31, wherein the mammal is a human.

33. A method for identifying one or more genetic markers associated with a psychiatric disorder, or a predisposition thereto, said method including the steps of:
(I) testing respective members of one or more pedigrees affected by said psychiatric disorder, using the method of claim 1;
(II) identifying members having said psychiatric disorder; and
(III) conducting genetic linkage analysis on the identified members to identify the or each genetic marker associated with said psychiatric disorder.

34. The method of claim 33, wherein the ambiguous moving image is a moving plaid.

35. The method of claim 34, wherein the psychiatric disorder is a mood disorder.

36. The method of claim 34, wherein the psychiatric disorder is schizophrenia or schizophrenia subtype.
